(19) United States
(12) Patent Application Publication
(10) Pub. No.: US 2026/0337174 A1
(43) Pub. Date: Oct. 29, 2026

(54) DECARBOXYLATION SYSTEM

(71) Applicant: Christopher W Blanch, Phoenix, AZ (US)

(72) Inventor: Christopher W Blanch, Phoenix, AZ (US)

(21) Appl. No.: 19/459,808

(22) Filed: Jan. 26, 2026

Related U.S. Application Data
(60) Provisional application No. 63/787,122, filed on Apr. 11, 2025.

(51) Int. Cl. C07D 311/80; B01J 19/00

(52) U.S. Cl. CPC C07D 311/80; B01J 19/0013

(57) ABSTRACT

Controlled-environment systems and methods for decarboxylating cannabinoid acids are disclosed. A sealed reaction vessel is operated under regulated temperature and above-atmospheric pressure conditions while an inert gas is circulated to inhibit oxidation and promote uniform heating. Independent control of temperature, pressure, and chemical atmosphere enables efficient conversion of cannabinoid acids while preserving volatile compounds, and controlled depressurization facilitates removal of dissolved gases.

### PRIORITY

This application claims priority to application number 63/787,122 filed on Apr. 11, 2025, which is incorporated herein by reference in its entirety.

### TECHNICAL FIELD

Embodiments of the present application generally relate to chemical processing technology. More particularly, the present application relates to methods, apparatuses, and systems for controlled-environment thermal decarboxylation of cannabinoid acids using regulated pressure, inert gas atmospheres, and precision temperature control.

### BACKGROUND

Cannabinoid-containing materials naturally include acidic precursor compounds, such as tetrahydrocannabinolic acid (THCa), which must undergo a decarboxylation reaction to form neutral cannabinoids such as delta-9-tetrahydrocannabinol (Δ9-THC). Decarboxylation is a thermally activated reaction in which a carboxyl group is removed from the molecule, releasing carbon dioxide and hydrogen as byproducts.

Conventional decarboxylation techniques commonly rely on open-air heating environments, ovens, or loosely controlled thermal processes. Such approaches frequently result in oxidation of cannabinoids, loss of volatile terpenes, non-uniform heating, localized overheating, and inconsistent conversion efficiency.

Vacuum-based decarboxylation systems have been employed to mitigate oxidation; however, vacuum conditions reduce boiling points and can accelerate volatilization of desirable compounds. Additionally, vacuum systems provide limited control over reaction kinetics and are often unsuitable for scalable commercial or industrial processing.

Accordingly, there exists a need for improved decarboxylation systems capable of independently controlling temperature, pressure, and chemical atmosphere while preserving volatile compounds and enabling repeatable, scalable operation.

### SUMMARY

Some embodiments of the present application provide controlled-environment decarboxylation systems and methods that independently regulate temperature, pressure, and chemical atmosphere within a sealed reaction environment.

In some embodiments, a pressurized reaction vessel is combined with inert gas circulation and precision thermal regulation to promote efficient and uniform decarboxylation while minimizing oxidation and loss of volatile constituents.

In some embodiments, controlled depressurization and optional post-heating steps facilitate removal of dissolved gases following decarboxylation, thereby producing a stabilized product suitable for downstream formulation, storage, or packaging.

### BRIEF DESCRIPTION OF THE DRAWINGS

FIG. 1 illustrates a schematic block diagram of a controlled-environment decarboxylation system, including an inert gas source, gas control components, a sealed reaction vessel, a heating assembly, a gas circulation module, a vent and pressure control module, and a control unit.

FIG. 2 illustrates an example control architecture in which a control unit coordinates heating, pressure regulation, and inert gas handling for a sealed reaction vessel.

FIG. 3 illustrates an example inert gas circulation subsystem configured to circulate an inert gas through a reaction vessel using one or more flow control valves, a filter, and a pump.

FIG. 4 illustrates an example method flow for controlled-environment decarboxylation, including material loading, vessel sealing, inert gas introduction, heating and pressurization, gas circulation, dwell time, controlled depressurization, and optional post-warming and outgassing.

FIG. 5 illustrates an example reaction vessel configuration including a heater, a vent, and gas inlet and outlet interfaces.

### DETAILED DESCRIPTION

Reference will now be made in detail to embodiments of the present disclosure, examples of which are illustrated in the accompanying drawings. The described embodiments are provided to explain the disclosed systems and methods and are not intended to be limiting.

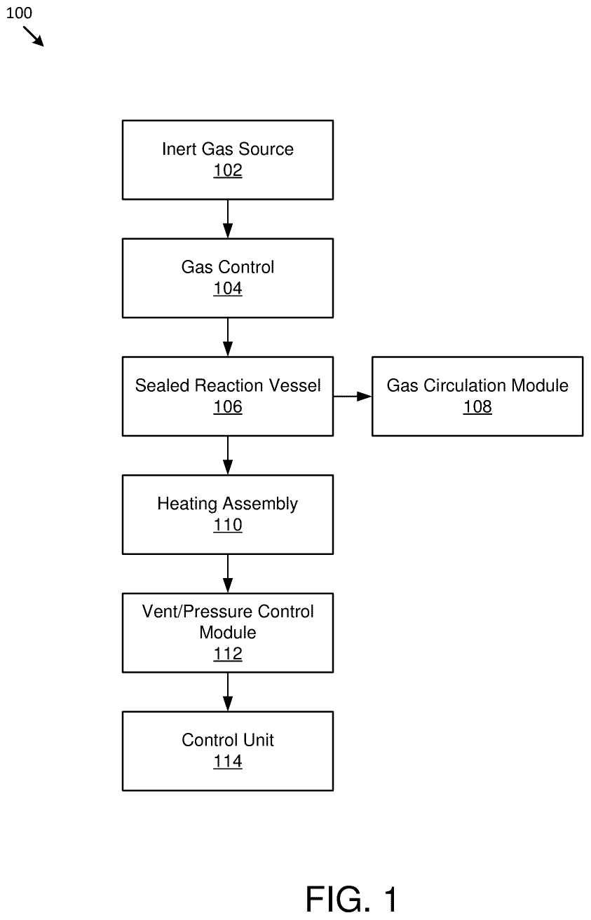

Referring to FIG. 1, a controlled-environment decarboxylation system 100 includes an inert gas source 102, a gas control module 104, a sealed reaction vessel 106, a gas circulation module 108, a heating assembly 110, a vent and pressure control module 112, and a control unit 114.

The sealed reaction vessel 106 is configured to receive cannabinoid-containing material in solid, liquid, or semi-solid form and to operate under elevated temperature and above-atmospheric pressure conditions. The reaction vessel 106 may be constructed from stainless steel, coated alloys, glass-lined materials, or other chemically resistant materials suitable for pressurized thermal processing.

The heating assembly 110 is thermally coupled to the reaction vessel 106 and may include resistive heating elements, jacketed fluid heating systems, induction heaters, or combinations thereof. In some embodiments, the heating assembly 110 maintains a temperature within a range suitable for decarboxylation, including temperatures between approximately 100 °F and 350 °F.

The inert gas source 102 supplies an inert gas selected from nitrogen, argon, carbon dioxide, or mixtures thereof. The gas control module 104 regulates delivery of the inert gas into the reaction vessel 106 and may include one or more valves, regulators, flow controllers, and check valves.

The gas circulation module 108 is configured to circulate the inert gas within the reaction vessel 106 to promote uniform heating and homogeneous reaction conditions. Circulation may occur continuously or intermittently during decarboxylation.

The vent and pressure control module 112 regulates internal vessel pressure and enables controlled depressurization following decarboxylation. In some embodiments, the pressure control module 112 maintains internal pressure above atmospheric pressure, including pressures between approximately 500 psi and 1500 psi.

The control unit 114 includes at least one processor and at least one memory storing executable instructions. The control unit 114 is configured to regulate operation of the heating assembly 110, the gas control module 104, the gas circulation module 108, and the vent and pressure control module 112 based on sensor feedback and stored decarboxylation recipes.

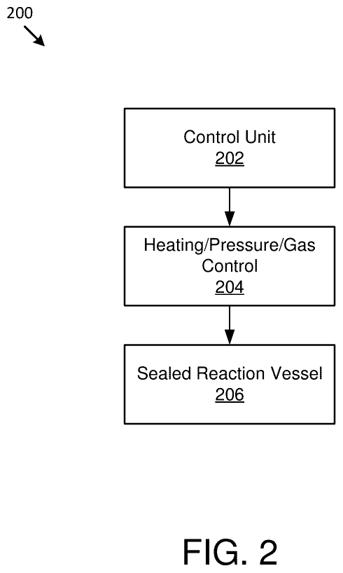

Referring to FIG. 2, a system 200 includes a control unit 202 operatively coupled to a heating, pressure, and gas control subsystem 204 and a sealed reaction vessel 206.

The control unit 202 receives input from one or more temperature sensors, pressure sensors, and flow sensors associated with the reaction vessel 206. Based on the received sensor data, the control unit 202 dynamically adjusts heating power, inert gas flow rate, circulation timing, and pressure setpoints.

In some embodiments, the control unit 202 executes stored decarboxylation recipes defining temperature profiles, pressure profiles, dwell durations, and depressurization sequences, thereby enabling automated and repeatable operation.

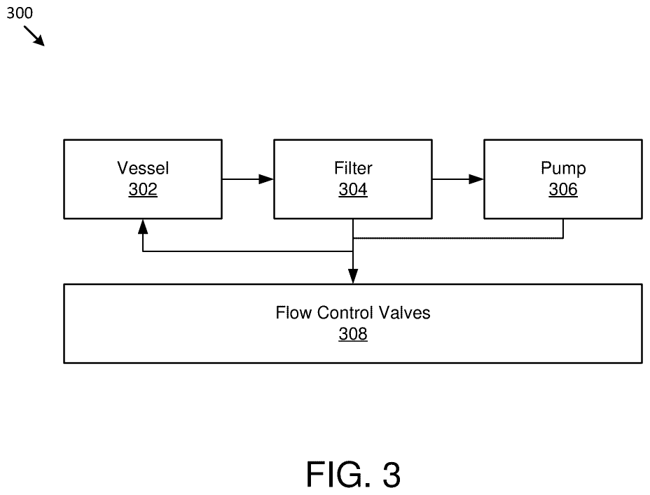

Referring to FIG. 3, an inert gas circulation subsystem 300 includes a vessel 302, a filter 304, a pump 306, and one or more flow control valves 308.

In some embodiments, the circulation subsystem 300 operates in a closed-loop configuration in which inert gas exiting the vessel 302 is filtered and recirculated back into the vessel. In other embodiments, the circulation subsystem 300 operates in an open-loop configuration in which inert gas is vented after passing through the vessel.

The circulation subsystem 300 may be configured to operate continuously or intermittently and may be controlled by the control unit to adjust flow rates based on temperature uniformity or process stage.

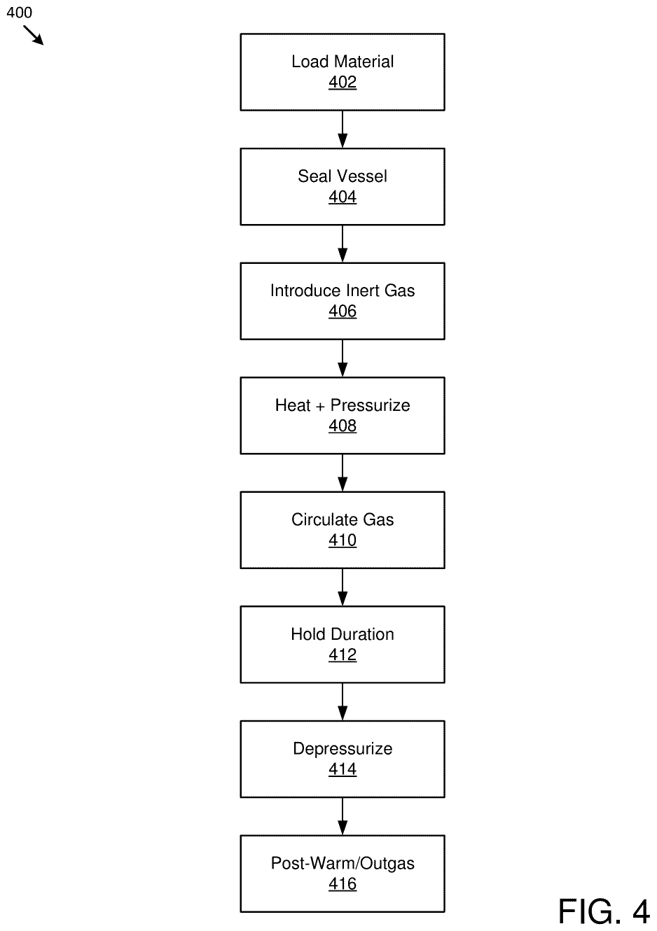

Referring to FIG. 4, a method 400 includes loading cannabinoid-containing material into a reaction vessel at step 402 and sealing the reaction vessel at step 404.

At step 406, an inert gas is introduced into the sealed reaction vessel to establish a non-oxidizing atmosphere. The inert gas displaces oxygen to inhibit oxidation of cannabinoids and preserve volatile terpenes.

At step 408, the material is heated to a decarboxylation temperature while pressure is maintained above atmospheric pressure. During step 410, the inert gas is circulated within the reaction vessel to promote uniform heating and reaction kinetics.

The system maintains the decarboxylation conditions for a predetermined duration at step 412, after which the vessel is depressurized in a controlled manner at step 414 to release dissolved gases.

In some embodiments, a post-warming or outgassing step 416 is performed following depressurization to further remove residual gases and stabilize the final product.

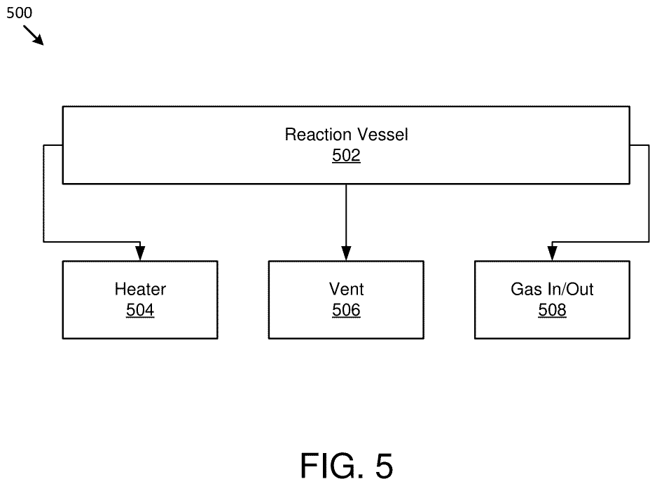

Referring to FIG. 5, a reaction vessel assembly 500 includes a reaction vessel 502, a heater 504 thermally coupled to the reaction vessel 502, a vent 506, and one or more gas inlet and outlet ports 508.

The vent 506 may include controllable valves, pressure relief devices, or safety interlocks configured to prevent overpressure conditions. The gas inlet and outlet ports 508 interface with the inert gas delivery and circulation subsystems.

The disclosed systems and methods may be implemented in batch, semi-continuous, or continuous processing configurations and may be scaled for laboratory, commercial, or industrial operation.

Although primarily described in connection with cannabinoid decarboxylation, the disclosed principles may be applied to other thermally activated chemical transformations requiring controlled temperature, pressure, and chemical atmosphere.

1. An apparatus for decarboxylating cannabinoid acids, comprising:
a sealed reaction vessel configured to contain cannabinoid-containing material; a heating assembly thermally coupled to the reaction vessel;
a pressure regulation unit configured to maintain internal pressure above atmospheric pressure during operation; and
an inert gas circulation system configured to introduce and circulate an inert gas within the reaction vessel.

2. The apparatus of claim 1, wherein the inert gas circulation system operates in a closed-loop configuration.

3. The apparatus of claim 1, further comprising a control unit configured to regulate temperature, pressure, and gas flow based on sensor feedback.

4. The apparatus of claim 1, wherein the inert gas is selected from nitrogen, argon, carbon dioxide, or mixtures thereof.

5. The apparatus of claim 1, wherein the heating assembly maintains a temperature between approximately 100 °F and 350 °F.

6. A method for decarboxylating cannabinoid acids, comprising:
placing cannabinoid-containing material into a sealed reaction vessel;
introducing an inert gas into the reaction vessel; heating the material to a decarboxylation temperature;
maintaining pressure above atmospheric pressure during heating; and
depressurizing the vessel following decarboxylation to release dissolved gases.

7. The method of claim 6, wherein the inert gas inhibits oxidation of cannabinoids.

8. The method of claim 6, wherein the pressure is maintained between approximately 500 and 1500 psi.

9. The method of claim 6, further comprising applying a post-heating step after depressurization.

10. The method of claim 6, wherein heating is controlled using a programmable temperature profile.

11. A system for controlled decarboxylation, comprising:
a reaction vessel;
a heating subsystem;
a pressure control subsystem;
a gas circulation subsystem; and
a controller configured to coordinate operation of the subsystems.

12. The system of claim 11, wherein the controller executes stored decarboxylation recipes.

13. The system of claim 11, wherein the system operates in a batch configuration.

14. The system of claim 11, wherein the system operates in a continuous configuration.

15. The system of claim 11, wherein the controller dynamically adjusts parameters based on sensor feedback.

16. The apparatus of claim 1, wherein the reaction vessel is configured for industrial-scale processing.

17. The apparatus of claim 1, wherein the reaction vessel includes safety interlocks.

18. The method of claim 6, wherein the cannabinoid-containing material includes tetrahydrocannabinolic acid.

19. The method of claim 6, wherein volatile terpenes are retained during decarboxylation.

20. The system of claim 11, wherein the system is configured for automated operation.
